(19) United States
(12) Patent Application Publication
(10) Pub. No.: US 2014/0128682 A1
(43) Pub. Date: May 8, 2014

(54) RETRACTOR TOOL

(71) Applicants: Oded Loebl, Tel Mond (IL); Avi Shterling, Yarkona (IL); Tzony Siegal, Moshav Shoeva (IL); Didier Toubia, Raanana (IL)

(72) Inventors: Oded Loebl, Tel Mond (IL); Avi Shterling, Yarkona (IL); Tzony Siegal, Moshav Shoeva (IL); Didier Toubia, Raanana (IL)

(73) Assignee: NLT SPINE LTD., Kfar saba (IL)

(21) Appl. No.: 14/131,912

(22) PCT Filed: Jul. 10, 2012
(86) PCT No.: PCT/IB2012/053516

§ 371 Date: Jan. 10, 2014

Related U.S. Application Data
(60) Provisional application No. 61/506,102, filed on Jul. 10, 2011.

(51) Int. Cl. A61B 17/02

(52) U.S. Cl. CPC A61B 17/025
USPC 600/206; 600/219

(57) ABSTRACT

An apparatus for tissue separation comprises a hollow retractor expandable in at least one dimension, the retractor having upper and lower guide surface portions for contacting respective upper and lower tissue surfaces; a first dilator having a distal portion configured to fit inside the upper and lower guide surface portions of the retractor so as to expand a distance between the upper and lower guide surface portions of the retractor and thereby separate the upper and lower tissue surfaces by a first distance; and a second dilator having a distal portion configured to fit inside the upper and lower guide surface portions of the retractor so as to expand a distance between the upper and lower guide surface portions of the retractor and thereby render the upper and lower tissue surfaces being separated by a second distance, the second distance greater than the first distance.

### FIELD AND BACKGROUND OF THE INVENTION

The present invention generally relates to apparatus and methods for tissue separation, and more particularly, to apparatus and methods for using an expanding retractor with successively larger dilator elements.

A common technique currently employed is passing the tip of a K-wire through tissues to the target location—then passing over the wire a succession of tubes of increasing diameter until reaching the optimal bore size. The drawback of this procedure is the ever increasing frictional trauma imposed on the tissue being separated. An additional shortcoming of the ‘tube’ system is that the final cross sectional shape is round.

In certain tissue environments, for instance when the target is the intervertebral disc space, approached from any angle, the above technique is not easily applicable as contiguous vital structures like nerves or blood vessels may be damaged in the process.

There is a compelling need for an improved method and apparatus for tissue separation, particular in spinal surgery.

### SUMMARY OF THE PRESENT INVENTION

One aspect of the present invention is an apparatus for tissue separation, comprising a hollow retractor expandable in at least one dimension, the retractor having upper and lower guide surface portions for contacting respective upper and lower tissue surfaces; a first dilator having a distal portion configured to fit inside the upper and lower guide surface portions of the retractor so as to expand a distance between the upper and lower guide surface portions of the retractor and thereby separate the upper and lower tissue surfaces by a first distance; and a second dilator having a distal portion configured to fit inside the upper and lower guide surface portions of the retractor so as to expand a distance between the upper and lower guide surface portions of the retractor and thereby render the upper and lower tissue surfaces being separated by a second distance, the second distance greater than the first distance.

A further aspect of the present invention is a method of tissue separation, comprising inserting a retractor into a tissue, the retractor having upper and lower guide surface portions that contact respective upper and lower tissue surfaces, the retractor expandable in at least one dimension so that a distance between the upper and lower guide surface portions is expandable; inserting a first dilator inside the retractor such that at least some of a length of the first dilator is placed between the upper and lower guide surface portions so as to increase a distance between the upper and lower guide surface portions and effectuate separation of the tissue by a first amount in at least one dimension, the first dilator comprising an elongated body; expanding the retractor in the at least one dimension by successively inserting a second dilator of greater size in the at least one dimension inside the retractor, the inserting performed such that at least some of a length of the second dilator is placed between the upper and lower guide surface portions so as to further increase the distance between the upper and lower guide surface portions thereby effectuating further separation of the tissue, the second dilator also comprising an elongated body.

A still further aspect of the present invention is a method of tissue separation, comprising inserting a retractor into a tissue, the retractor having upper and lower guide surface portions that contact respective upper and lower tissue surfaces, the retractor expandable in at least one dimension so that a distance between the upper and lower guide surface portions is expandable; inserting a first dilator inside the retractor such that at least some of a length of the first dilator is placed between the upper and lower guide surface portions so as to increase a distance between the upper and lower guide surface portions and effectuate separation of the tissue by a first distance, the first dilator comprising an elongated body; expanding the retractor in at least one dimension by inserting a second dilator of greater size in the at least one dimension inside the retractor without removing the first dilator, the inserting performed such that at least some of a length of the second dilator is placed between the upper and lower guide surface portions so as to further increase the distance between the upper and lower guide surface portions thereby effectuating further separation of the tissue by a greater distance.

A yet still further aspect of the present invention is a method of sizing a limit of tissue separation, comprising (a) assessing a quality of tissue being separated by (i) inserting a retractor into a tissue, the retractor having upper and lower guide surface portions that contact respective upper and lower tissue surfaces, the retractor expandable in at least one dimension so that a distance between the upper and lower guide surface portions is expandable, and by (ii) inserting a first dilator inside the retractor such that at least some of a length of the first dilator is placed between the upper and lower guide surface portions so as to increase a distance between the upper and lower guide surface portions and effectuate separation of the tissue by a first distance; and (b) sizing/assessing a limit to which the tissue is separable by expanding the retractor in at least one dimension by successively inserting a further dilator of successively greater size in the at least one dimension inside the retractor, the inserting performed such that at least some of a length of each of the further dilators is placed between the upper and lower guide surface portions so as to further increase the distance between the upper and lower guide surface portions thereby effectuating further separation of the tissue in successively greater amounts.

A still further aspect of the present invention is an apparatus for tissue separation, comprising a hollow retractor expandable in at least one dimension, the retractor having upper and lower guide surface portions for contacting respective upper and lower tissue surfaces; a dilator having a distal portion configured to fit inside the upper and lower guide surface portions of the retractor so as to expand a distance between the upper and lower guide surface portions of the retractor and thereby separate the upper and lower tissue surfaces by a first distance.

A yet still further aspect of the present invention is a method of using a retractor for an insertion of an element into a human body, comprising inserting a retractor into a tissue, the retractor having upper and lower guide surface portions that contact respective upper and lower tissue surfaces, the retractor expandable in at least one dimension so that a distance between the upper and lower guide surface portions is expandable; inserting a dilator inside the retractor such that at least some of a length of the first dilator is placed between the upper and lower guide surface portions so as to increase a distance between the upper and lower guide surface portions and effectuate separation of the tissue by a first amount in at least one dimension; forming a passageway inside the retractor by removing the dilator; and inserting an implant through the passageway and into the human body.

Another aspect of the present invention is a method of using a retractor for an insertion of an element into a human body, comprising inserting a retractor into a tissue, the retractor having upper and lower guide surface portions that contact respective upper and lower tissue surfaces, the retractor expandable in at least one dimension so that a distance between the upper and lower guide surface portions is expandable; inserting a dilator inside the retractor such that at least some of a length of the first dilator is placed between the upper and lower guide surface portions so as to increase a distance between the upper and lower guide surface portions and effectuate separation of the tissue by a first amount in at least one dimension; forming a passageway inside the retractor by removing the dilator; and inserting a surgical tool through the passageway and into the human body.

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is an isometric view of a distal portion of retractor, in accordance with one embodiment of the present invention;

FIG. 2 is an isometric view of a distal portion of tissue separator, in accordance with one embodiment of the present invention;

FIG. 3 is an isometric view of a distal portion of a tissue separator of FIG. 2, with the core element of the first dilator removed and the side support element of the first dilator remaining, in accordance with one embodiment of the present invention;

FIG. 4 is an isometric view of a distal portion of the tissue separator of FIG. 1-3, with a core element of a larger second dilator and the side support element of the first dilator remaining, in accordance with one embodiment of the present invention;

FIG. 5 is an isometric view of a distal portion of the tissue separator of FIG. 4, with a core element of the larger second dilator remaining and the side support element of the first dilator removed, in accordance with one embodiment of the present invention;

FIG. 6 is an isometric view of a distal portion of the tissue separator of FIG. 5, with the side support element of the second larger dilator inserted alongside the core element of the second larger dilator, in accordance with one embodiment of the present invention;

FIG. 6A is an isometric view of a retractor, in accordance with one embodiment of the present invention;

FIG. 7 is an isometric view from the side of a retractor with spring arms, in accordance with one embodiment of the present invention;

FIG. 7A is a schematic view of a retractor with spring arms, in accordance with a different embodiment of the present invention;

FIG. 8 is an isometric view from the top and side of the retractor with spring arms of FIG. 7, in accordance with one embodiment of the present invention;

FIG. 9 is an isometric view from the bottom of the retractor with spring arms of FIG. 7, in accordance with one embodiment of the present invention;

FIG. 10A is an isometric view of the retractor of FIG. 7 without a dilator inserted, in accordance with one embodiment of the present invention;

FIG. 10B is an isometric view of the retractor of FIG. 7 with a dilator inserted, in accordance with one embodiment of the present invention;

FIG. 10C is an isometric view of the retractor of FIG. 7 with the dilator further inserted, in accordance with one embodiment of the present invention;

FIG. 10D is an isometric view of the retractor of FIG. 7 with the dilator inserted and causing retraction of the retractor aims, in accordance with one embodiment of the present invention;

FIG. 11 is an isometric view from the side and top of a retractor with hinged arms, in accordance with one embodiment of the present invention;

FIG. 12 is an isometric view from the side of the retractor with spring arms of FIG. 11, in accordance with one embodiment of the present invention;

FIG. 13 is an isometric view from the top of the retractor with spring arms of FIG. 11, in accordance with one embodiment of the present invention;

FIG. 14A is an isometric view of the retractor of FIG. 11 without a dilator inserted, in accordance with one embodiment of the present invention;

FIG. 14B is an isometric view of the retractor of FIG. 11 with a dilator inserted, in accordance with one embodiment of the present invention;

FIG. 14C is an isometric view of the retractor of FIG. 11 with the dilator further inserted, in accordance with one embodiment of the present invention;

FIG. 14D is an isometric view of the retractor of FIG. 11 with the dilator fully inserted, in accordance with one embodiment of the present invention;

FIG. 15A is an isometric view of a further embodiment of a tissue separator of the present invention including a retractor having flaps, in accordance with one embodiment of the present invention;

FIG. 15B is an isometric rear view of the tissue separator of FIG. 15A, in accordance with one embodiment of the present invention;

FIG. 16A is an isometric view of the tissue separator of FIG. 15A with the dilator forward enough to move the flaps open, in accordance with one embodiment of the present invention;

FIG. 16B is an isometric rear view of the tissue separator in the position of FIG. 16A, in accordance with one embodiment of the present invention;

FIG. 17A is an isometric view of the tissue separator of FIG. 15A with the dilator further forward, in accordance with one embodiment of the present invention;

FIG. 17B is an isometric rear view of the tissue separator in the position of FIG. 17A, in accordance with one embodiment of the present invention;

FIG. 18 is an isometric view of a tissue separator inserted between two adjacent vertebrae;

FIG. 19 is a flow chart of a method, in accordance with one embodiment of the present invention;

FIG. 20a is an isometric view of a curved cross-section for upper of lower guide surface portions of a retractor, in accordance with one embodiment of the present invention; and

FIG. 20b is an isometric view of a flat cross-section for upper of lower guide surface portions of a retractor, in accordance with one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a method and apparatus for tissue separation. The tissue separation may be necessary as part of a surgical procedure or operation, for example during a discectomy, for example where it is necessary to increase the gap between two vertebrae. A retractor, expandable in at least one dimension, may be inserted into the area of the tissue, for example between discs of a spine. The retractor may have a distal guide surface or surfaces (meaning distal in relation to the surgeon which in this case may mean inside the subject's spine) including upper and lower guide surface portions that may make contact with respective upper and lower tissue surfaces. In a preferred embodiment, after the retractor is in place, one may insert into the retractor a first dilator having a distal portion that can forcibly fit inside the upper and lower guide surface portions of the retractor so as to expand the distance between the upper and lower guide surface portions of the retractor and thereby separate the upper and lower tissue surfaces by a first distance. In one preferred embodiment, the first dilator is removed from the retractor and a further dilator that is larger in the dimension that the tissue dilator is expanded, for example in height, is then inserted into the retractor. In a second preferred embodiment, the first dilator remains in the retractor while a further second dilator having a larger height in the dimension that the retractor expands in can be inserted forcibly inside the retractor. This may expand the distance between the upper and lower guide surface portions of the retractor and thereby render the upper and lower tissue surfaces being separated by a second distance greater than the first distance. Passage of the dilators may optionally be a tissue sampling procedure as well as a sizing dilation step. Reaching the final dilation may provide information regarding the size of the disc space, consequent preparatory tools (or other tools) as well as the size of the implant that is to be surgically inserted, for example between the vertebrae. The upper and lower guide surface portions of the retractor may, in some preferred embodiments, effectively act like a shoe horn, displacing the adjacent tissue as successive dilators are advanced between the upper and lower guide surface portions while greatly reducing frictional trauma to the tissue. The retractor depicted herein may be square in its cross-sectional shape, but it can assume any other shape, regular or irregular, symmetric or asymmetric. The retractor can be used for tissue separation anywhere in the body of humans and non-humans. For the sake of example, the embodiment presented is one of separating tissues on the way to and within the intervertebral disc. In a typical application, corresponding to a method according to the present invention, a k-wire is inserted within the disc space and its position is verified by a C-arm fluoroscopy. The retractor is passed over the k-wire with the thinner distal end of the retractor penetrating the annulus and its position determined by fluoroscopy. The penetration point through the annulus is at the discretion of the user, all around the annulus, 360 degrees. The retractor may be held secure by a fixator device attached to the operating table—or may be fixed to vertebral screws (or other devices such as spinous process fixation devices)—or may be held manually by the user—or by other means. Once the retractor has been positioned within the tissue space (disc space in the case of spinal surgery), dilators may be sequentially introduced into it to enlarge at least part of its length the opening and allow passage of instruments, implants and/or materials (such as fusion inducing materials) inwardly and/or passage outwardly (i.e. disc tissue). The proximal portion of the retractor arms typically remains out of the body of the patient/subject and its height may remain constant. In the case of disc space procedures, this height typically reaches about 14 mm but may differ according to the specific application of the device.

In contrast to prior art retractors, in which successive hollow tubes may be placed on a K-wire in order to separate tissue, thereby causing trauma to the tissue as each successive tube is dragged along the tissue in order to reach its place, the method and apparatus of the present invention may be used to separate the affected tissue without causing repeated trauma. Successive dilators may be inserted through a hollow retractor expandable in at least one dimension. In this way, only the retractor need contact the tissue. The dilators need not contact the tissue. By inserting successive dilators, the tissue to be separated may be sized without having to successively pass tubes past the tissue. There may be no relative motion causing friction between the retractor and the tissue with which it comes in contact. The progressive increase in one or more dimensions from within the inside of the retractor eliminates the successive passage of traumatizing tubes on the outside of the retractor. In further contrast to the prior art, in which the cross-section of the successive tubes may be circular, the retractor may have a generally square cross-section. In contrast to the prior art, passage of the dilators increasing in size, may offer vital information to the user imparted by tactility, i.e. assessment of disc tissue quality whether dry and fibrotic or plump and soft. It also allows the user to feel the limits of the stretch that can be applied to the surrounding soft tissues of the motion segment and so avoid excessive dilation and endplate damage and/or soft tissue damage (tearing of muscle or ligamentous tissue such as the anterior longitudinal ligament).

The principles and operation of an apparatus and method for a retractor tool with successive dilators according to the present invention may be better understood with reference to the drawings and the accompanying description.

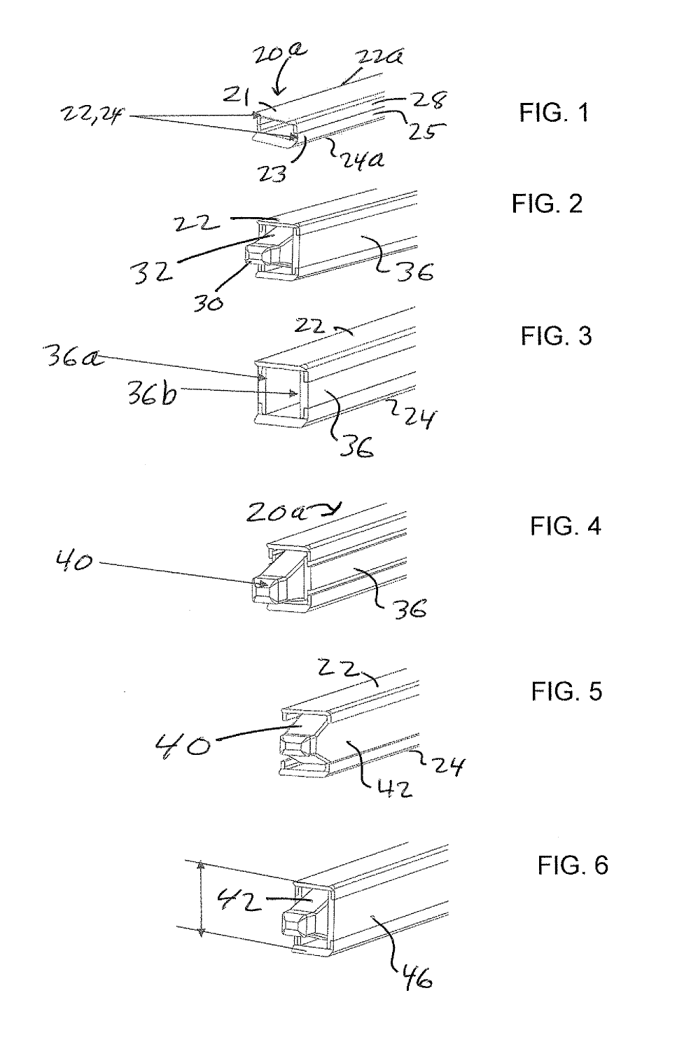

FIG. 1 through FIG. 6 depict successive stages of tissue separation using an apparatus 10 of the present invention, in accordance with a preferred embodiment. In each of these figures the more proximal portion of the tissue separator apparatus is not shown. In one preferred embodiment, not all of the length of the retractor is adjustable in the at least one dimension, for example in height. Rather, only the distal part of the retractor is adjustable in height. The retractor may have a guide portion (also called a guide surface portion) at its distal end 20a, meaning the portion that is configured to contact the tissue. In one preferred embodiment, the guide portion of the retractor comprises two guide portions (also called guide surface portions).

As shown from FIGS. 1-6, tissue separator 10 may comprise a hollow retractor 20 expandable in at least one dimension, the retractor having upper and lower guide surface portions for contacting respective upper and lower tissue surfaces. The phrase “upper and lower” guide surface portions or guide surfaces and the individual terms “upper” and “lower” in these phrases should be understood as relative terms in any appropriate dimension, including but not limited to the height dimension. They could, for example, also refer to a width dimension. Tissue separator 10 may also comprise a first dilator 30 having a distal portion configured to fit inside the upper and lower guide surface portions of the retractor so as to expand a distance between the upper and lower guide surface portions of the retractor and thereby separate the upper and lower tissue surfaces by a first distance.

The tissue separator 10 may also include a second dilator 40 having a distal portion configured to fit inside the upper and lower guide surface portions of the retractor so as to expand a distance between the upper and lower guide surface portions of the retractor and thereby render the upper and lower tissue surfaces being separated by a second distance, the second distance greater than the first distance. The second dilator may be larger than the first dilator in at least one dimension, for example in the height dimension, which in the context of spinal surgery, can be called the cephalad-caudad dimension. The dilators may be made of any biocompatible material(s), including but not limited to metallic material(s), polymeric material(s) or other or a combination of such materials.

In one preferred embodiment, the retractor 20 is defomable in at least a distal portion of a length of the retractor 20 to accommodate dilators that are successively larger in at least one dimension. For example, in this regard, the distal portion may be the last 10%, or in other preferred embodiments, at least 50% of the length of the retractor, or in some preferred embodiments, at least the upper and lower guide surface portions of the retractor.

In any case, the length of the retractor arm that deforms does not necessarily have to be symmetric to the other retractor arm. For example, one retractor may be fixed and another retractor may be the component that performs all the retraction. Optionally one retractor may deform the distal part to a certain amount while the other retractor deforms to a different amount (either more or less than the first retractor.

The retractor 20, or at least the upper and lower guide surface portions, may be deformable as a result of being made from plastic and/or superelastic material. For example, the upper and lower guide surface portions may be formed from Nitinol superelastic materials, allowing them to smoothly deform to accommodate successively wider dilators advancing between the upper and lower guide surface elements, and then return to their original form. In other embodiments, the guide surface portions may be plastically deformed during insertion of the dilators.

In a second preferred embodiment shown in FIGS. 1-6, the retractor 20 comprises retractor arms 22a, 24a defined by upper and lower U-shaped walls 22, 24, as shown in FIG. 1. In the initial position in FIG. 1, when no dilator has been inserted, the retractors arms 22a, 24a may contact one another, i.e. the legs 28, 25 of the U-shaped walls 22, 24 may contact one another. As can be seen, the cross-sectional shape of retractor 20 (and of tissue separator 10 as a whole including dilator 30) may be generally rectangular, and in some cases square or generally square, to facilitate correct orientation. “Generally” rectangular or “generally” square includes rectangular or square shapes where the corners are rounded, chamfered or deviate from a perfect rectangle or square. In addition, the term “generally rectangular” should be understood as including structures whose surface(s) include open spaces, such as open walls. As seen from FIGS. 1-6, the cross-sectional shape may be generally rectangular at a distal end 20a (as measured relative to the user not the patient) of the retractor 20. The cross-sectional shape of the upper and lower guide surface portions may extend across a majority of a length or in other preferred embodiments a full length of the retractor. In a preferred embodiment, the height as measured between the upper and lower guide surface portions 21, 23 of retractor arms 22a, 24a may generally be the same as the height of more proximal portions of retractor arms. In this preferred embodiment, the retractor 20 may have a fixed distance between upper and lower walls and the upper and lower guide surface portions 21, 23 may be expandable from one or more positions narrower than the fixed distance.

As shown in FIG. 2 (first dilator) and FIGS. 4-6 (second dilator), each of the first and second dilators may comprise a core element and a support element, the support element located on a side of the core element. For example, first dilator 30 may include core element 32 and support element 36 while second dilator 40 may include core element 42 and support element 46.

First dilator 30 may comprise a core element 32 (sometimes called a first core element since it is the core element of the first dilator 30) that has at least a top surface 34 and a bottom surface 35, and may comprise a support element 36 (also sometimes referred to as a first support element 46 since it is the support element for the first dilator 30) that may have at least one side wall 36a, and in a preferred embodiment two (left and right) side walls 36a, 36b. Similarly, second dilator 40 may comprise a core element 42 (sometimes called a second core element since it is the core element of the second dilator 40) that has at least a top surface 44 and a bottom surface 45, and may comprise a support element 46 (also sometimes referred to as a second support element 46 since it is the support element for the second dilator 40) that may have at least one side wall 46a, and in a preferred embodiment two (left and right) side walls 46a, 46b.

As can be appreciated from FIGS. 3-6, the core element 32 and/or 42 may be removable from the retractor without the support element having to be removed from the retractor. For example, FIG. 3 depicts a stage in which core element 32 of first dilator 30 has been pulled out and the side support elements 36a, 36b remains in order to support the structure of retractor 20, i.e. so that retractor arms 22a, 24a do not collapse to contact one another as in FIG. 1. In this stage a working tunnel has been created for surgical. (or other) use. In some other preferred embodiments, dilator core element 32, 42 is hollow and the working tunnel is available anyway.

FIG. 4 depicts the stage after a core element 42 of a larger size dilator, i.e. second dilator 40 has been inserted through retractor arms 22a, 24a. Accordingly, it depicts a stage where inside the retractor 20 there is a core element 42 of second dilator 40 that is together in the retractor with a support element 36 of a different dilator (first dilator 30), the different dilator being smaller in size than the second dilator 40. The core element 42 has a top and bottom surface. The support element 36 has at least one side wall.

In some preferred embodiments, the support element may be removable from the retractor without the core element having to be removed.

First dilator 30 may have a first core element and a first support element of a first size and second dilator 40 may have a second core element and a second support element of a second size. For example, first core element 32 and first support element 36 may both be 7 mm in height (as measured by the distance between retractor arms 22a, 24a) whereas second core element 42 and second support element 46 may be 9 mm in height (as measured by the distance between retractor arms 22a, 24a). Typically, for any dilator its core element and its support element may be equal in height, although there could be preferred embodiments in which the core element of a dilator is slightly larger or slightly smaller (for example plus or minus 1% or 2% or 4% or 5% or 7% or 10% or 15% or more) than the support element (i.e. side walls) of that dilator.

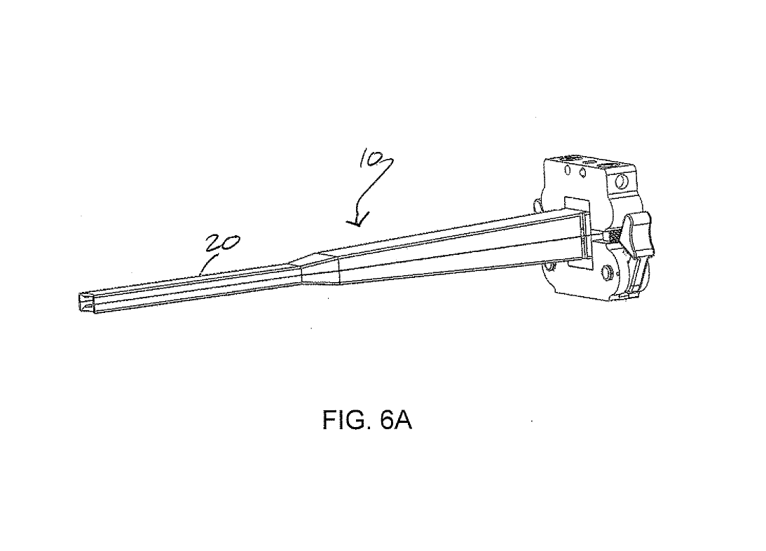

FIG. 5 depicts a stage in which the support element 36, i.e. the side support walls 36a, 36b of the first dilator 30 have been pulled out of retractor 10. Accordingly, the core element 42 of second dilator 40 is supporting the upper U-shaped walls 22 of retractor arm 22a and preventing it from contacting lower U-shaped walls 24. FIG. 6 depicts the stage in which the second support element 46, i.e. the side support walls 46a, 46b, of second dilator 40, has been inserted. This completes the cycle of expanding the tissue separator 10. The stages depicted in FIGS. 3 through 6 may be repeated as many times as appropriate to effectuate further expansion of retractor arms 22a, 24a, for example using a third dilator (not shown) whose core element and support element are of a third size. For example, using the previously mentioned sizes of the tissue separator, tissue separator 10 may be further expanded in at least one dimension, i.e. height, from 9 mm to 11 mm. As a further example, a still further expansion may be done with a fourth dilator (not shown) whose core element and support element are of a fourth size to further expand the tissue separator 10 from the at least one dimension, i.e. height from 11 mm to 13 mm. It should be clearly understood that these numbers for the height dimension, 7 mm, 9 mm, 11 mm, 13 mm are of course mere examples of height and expansion of height. Any other suitable numerical increments or sizes may be used.

It is noted that the length of any of the dilators (i.e. 30, 40) in the method and apparatus of the present invention may be greater than, equal to, or less than the length of the retractor 20. Furthermore, any of the dilators may be positioned relative to the retractor so that the dilator extends distally beyond the retractor. The purpose of having a dilator extend beyond the retractor may be so that (a) the dilator may reach/extend beyond the retractor tips and pierce/dilate/expand/manipulate tissue that is exterior to the retractor tips, and/or (b) the dilation may be very gradual (i.e. dilator tip may have a small angle).

The apparatus for tissue separation described in FIGS. 1-6, may also be described more generally as a combination of a retractor and a dilator. Tissue separator 10 may thereby comprise a hollow retractor 20 expandable in at least one dimension, the retractor having upper and lower guide surface portions 21, 23 for contacting respective upper and lower tissue surfaces. The apparatus may also include a dilator 30 having a distal portion configured to fit inside the upper and lower guide surface portions of the retractor so as to expand a distance between the upper and lower guide surface portions of the retractor and thereby separate the upper and lower tissue surfaces by a first distance.

The apparatus may include a mechanism for maintaining the hollow retractor in a retracted position with a set distance between the upper and lower guide surface portions when the dilator is removed before being replaced with a dilator of a different size. In some preferred embodiments, for example FIGS. 1-6A, this mechanism for maintaining the retractor is comprised of the retractor, for example the retractor arms, having upper and lower U-shaped walls and may be further comprised of the dilator having a core element 32 and a support element 36 that may have at least one side wall, wherein the support element 36 may be located on a side of the core element 32. The core element 32 may have a top and bottom surface. The core element 32 may be removable from the retractor without the support element having to be removed from the retractor.

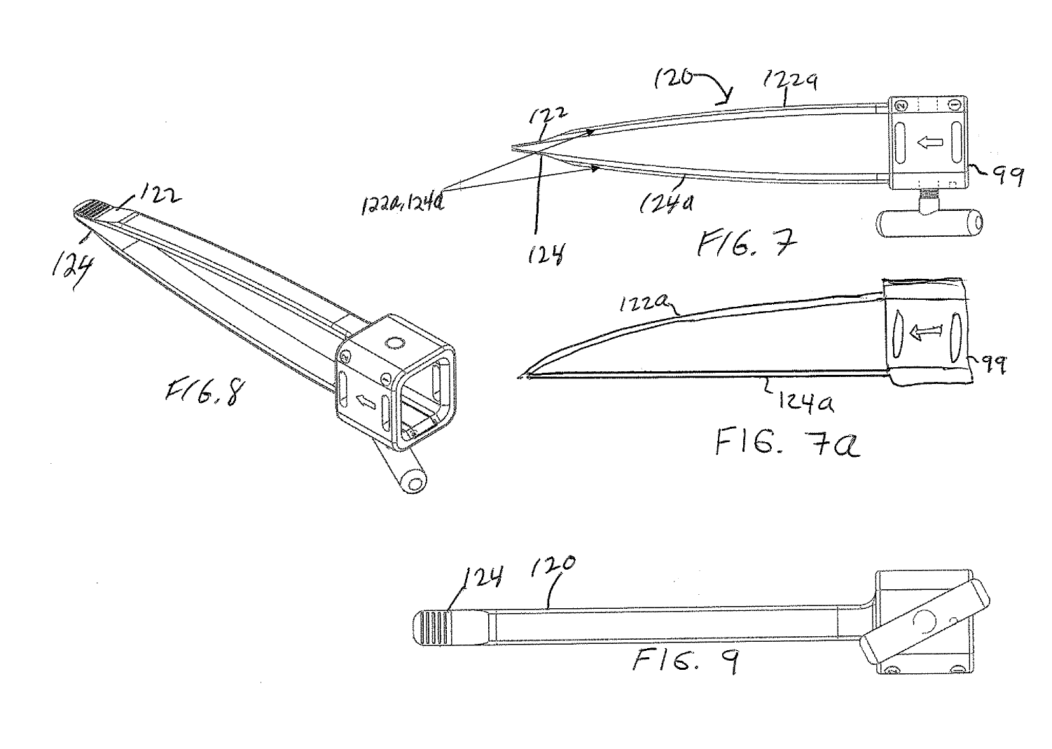

In another preferred embodiment, the mechanism for maintaining the retractor in a retracted position may be a hinge mechanism (for example hinge mechanism 250 as seen in FIG. 12) at a proximal end of the retractor for fixing a position of one or more retractor arms. This is described in FIGS. 7-14D. In a still further preferred embodiment, the mechanism for maintaining the retractor is a retracted position may be a ratchet mechanism (not shown).

As can be seen, the retractor of the present invention (20, 120, 220, 320) is expandable in a height dimension. In some preferred embodiments, the retractor is expandable in a height dimension and in a width dimension. This is necessary in certain surgical environments. Enlargement of the opening may vary taking into consideration the point of penetration: in the case of PLIF or TLIF procedures, the opening preferably gains only in height (in the sagittal plane, (i.e., the cephalad-caudad dimension)) as sideways/lateral widening of the entrance may compromise the adjacent nerve root and/or dura. The width of the retractor may be fixed and may not change all along the instrument. If the penetration is through the anterior annulus as in ALIF (or through soft tissue), widening may be concomitantly done sideways as well. If the widening is in more than one direction, the widening may be simultaneous in the plurality of directions or each direction widened in sequence.

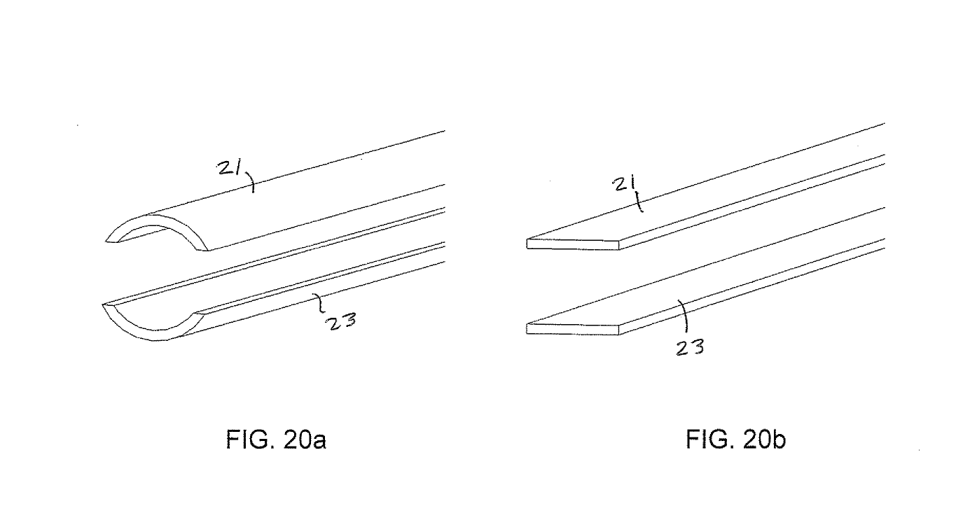

As seen in FIGS. 1-6, the guide surface portions of retractor arms 22a, 24a may be comprised of opposing guide surfaces. It should be understood that the guide surface portions in any preferred embodiment of the present invention may be flat (see, e.g. FIG. 20B), may be rounded (see, e.g. FIG. 20A) or may be a combination of the two or other possibilities (i.e. chamfered for example). Although FIGS. 20a, 20b relate to guide surface portions 21, 23 of retractor 20, the range of shapes applies to all preferred embodiments. The guide surface portions may also be parallel to one another, although this is not required in all cases. In certain preferred embodiments, the guide surface may be a single continuous surface, for example conical, and the upper and lower guide surface portions may be selected segments or portions of this single continuous surface.

In any of the embodiments of the method or apparatus of the present invention, in expanding the distance between the upper and lower surface guide portions, the upper and lower surface guide portions may be moved symmetrically or asymmetrically and may or may not have an equal share in performing the retraction. First of all, the length of one retractor arm that deforms or moves against the tissue may differ from the length of the other retractor arm that deforms or moves against the tissue. For example, the upper retractor arm's upper surface guide portion may have more of its length deform or move than the lower retractor arm's lower surface guide portion. Second, the quantity of the retraction movement may be more or less for one retractor arm's surface guide portion than for another. One extreme example of this, which may appear in certain preferred embodiments, is, as shown in FIG. 7A, where one retractor arm (here, the lower retractor arm) remains flat and the other retractor arm converges down to the distal tip of the first retractor arm prior to retraction. In this case, all of the tissue separation will be performed by retraction of the upper surface guide portion forming part of the upper retractor arm, with the other arm retractor arm remaining fixed. In a different preferred embodiment, one retractor arm's surface guide portion may retract or deform to a certain amount while the other retractor deforms to a different amount (either more or less than the first retractor arm's surface guide portion.

In a further preferred embodiment shown in FIGS. 7-10D, the tissue separator 100 includes a retractor 120 and a dilator of a first size. Retractor 120 has upper and lower spring arms 122a, 124a including upper and lower guide surface portions 122, 124 thereof. The spring arms 122a, 124a may be rigidly connected to the base 99, or connected by a moveable axis, or similar means. Each spring arm 122a, 124a may be connected to the base 99 by the same or different mechanism. For example, one arm may be rigid (without elastic/spring features) while the other arm bends and or moves/rotates. As shown in FIG. 7 and FIG. 8, the tips 127, 129 of the upper and lower guide surface portions 122, 124 of retractor 120 may be urged to contact one another before insertion of the first dilator 130 or the second dilator 140 inside retractor 20. In other words, upper and lower spring arms 122a, 124a converge at a tip 127, 129, the tip comprising part of the upper and lower guide surface portions 122, 124.

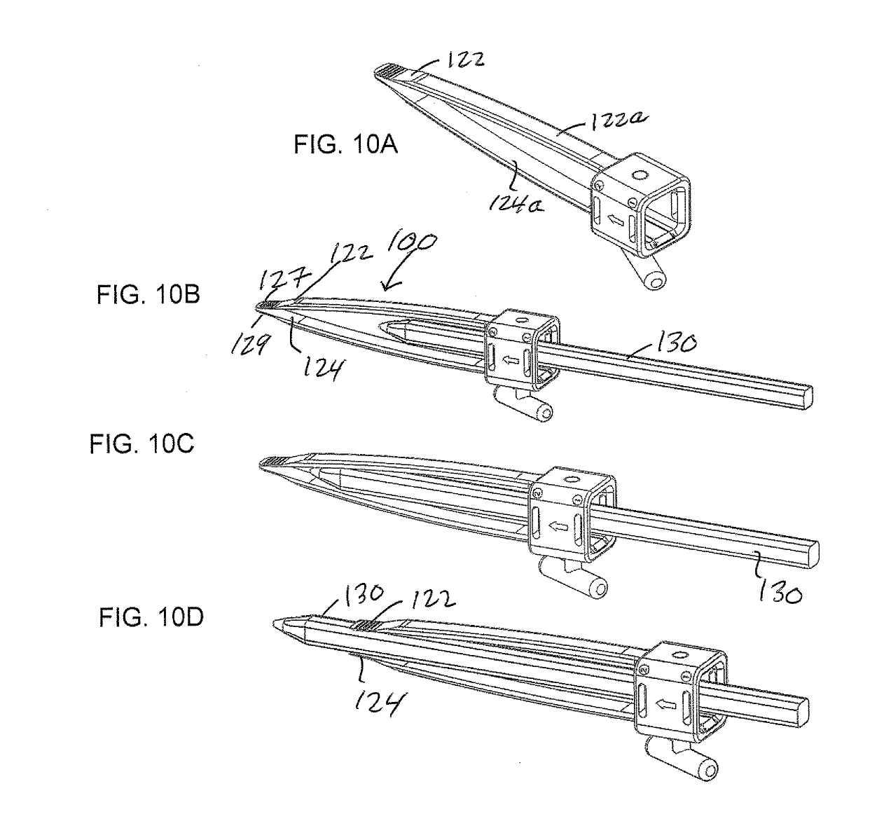

FIGS. 10A through 10D shown various stages of insertion of a dilator into the retractor 120 of tissue separator 100. In FIG. 10A, the retractor 120 has been inserted into the tissue of the subject but no dilator has been inserted. In FIG. 10B, first dilator 130 has been partially inserted but not to the point where the upper and lower guide surface portions of retractor 120 have been retracted. In FIG. 10C, the first dilator 130 is about to reach the upper and power guide surface portions. FIG. 10D shows the stage where first dilator 130 has expanded the distance between the upper and lower guide surface portions. The dilators may or may not be hollow. To expand the retraction, the first dilator is removed and a larger second dilator 140 is inserted. In the converse situation, a larger dilator is removed and a smaller dilator is inserted.

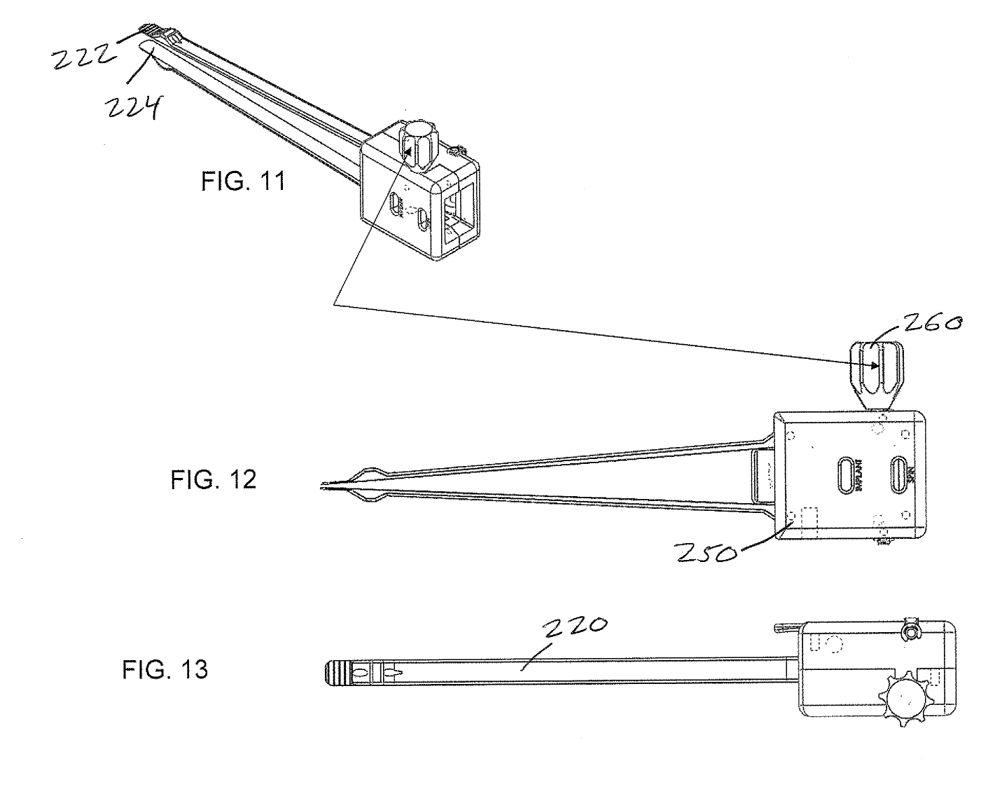

In a still further preferred embodiment of a tissue separator 200 shown in FIGS. 11-14D, the tissue separator 200 includes a retractor 220 and a first dilator 230. A hinge mechanism 250 at a proximal end of the retractor 220 may be used for initial retraction of the upper and lower guide surface portions 222, 224 to a distance that may allow upper and lower guide surface portions 222, 224 to abut the respective upper and lower tissue surfaces 11, 12 without actually retracting the tissue surfaces. For example, the hinge mechanism 250 may be controlled by a screw mechanism 260 that controls how far apart the upper and lower guide surface portions of the retractor arms are. The screw mechanism 260 may also set a maximum retraction of the upper and lower guide surface portions 222, 224, i.e. the maximum retraction distance of the upper and lower guide surface portions 222, 224 achievable with a particular first dilator or a particular second or further dilator. For example, the screw mechanism 260 may be used to retract the retractor arms to a distance of 4 mm so that the upper and lower guide surface portions abut the upper and lower tissue surfaces. The screw mechanism may also be set to a maximum distance of 7 mm to accommodate first dilator 230.

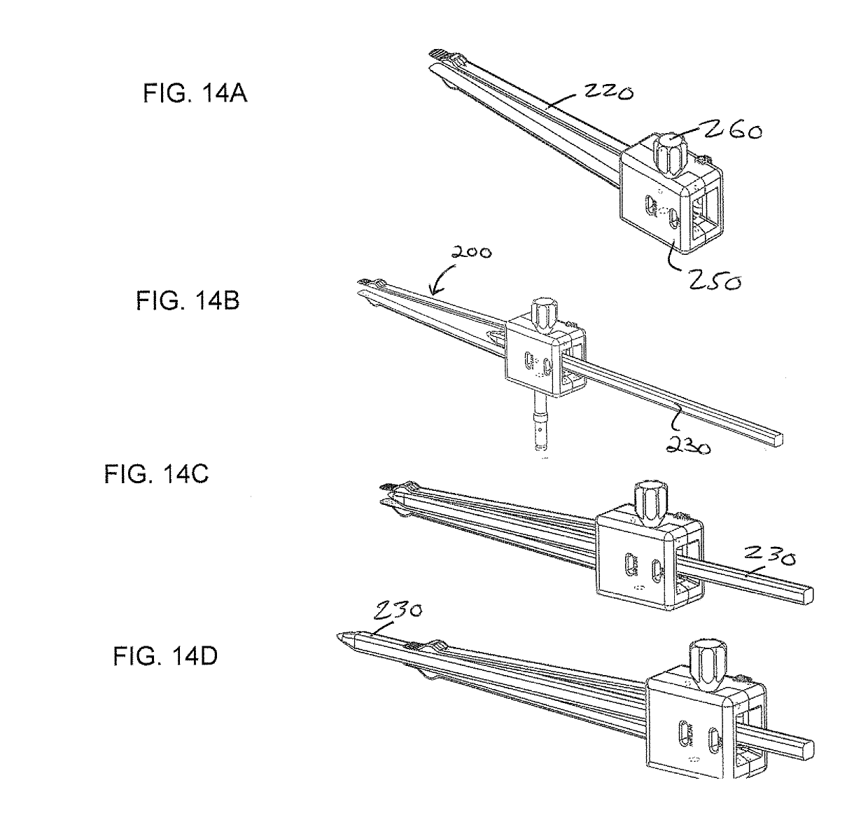

FIGS. 14A through 14D shown various stages of insertion of a dilator into the retractor 220. In FIG. 14A, the retractor 220 has been inserted into the tissue of the subject in its closed position with the tips touching (and no dilator has been inserted). After insertion into the tissue, the retractor arms are opened with the screw mechanism by turning the knob to reach an initial retraction position seen in FIG. 14A. In FIG. 14B, first dilator 230 has been partially inserted but not to the point where the upper and lower guide surface portions 222, 224 of retractor 220 have been further retracted (fix this figure the tips should be open like in 14A). In FIG. 14C, the first dilator 230 is about to reach the upper and power guide surface portions 222, 224. FIG. 14D shows the stage where first dilator 230 has expanded the distance between the upper and lower guide surface portions 222, 224 to its maximum dilation position previously set by the screw mechanism. To expand the retraction, the first dilator is removed and a larger second dilator 240 is inserted. In the converse situation, a larger dilator is removed and a smaller dilator is inserted.

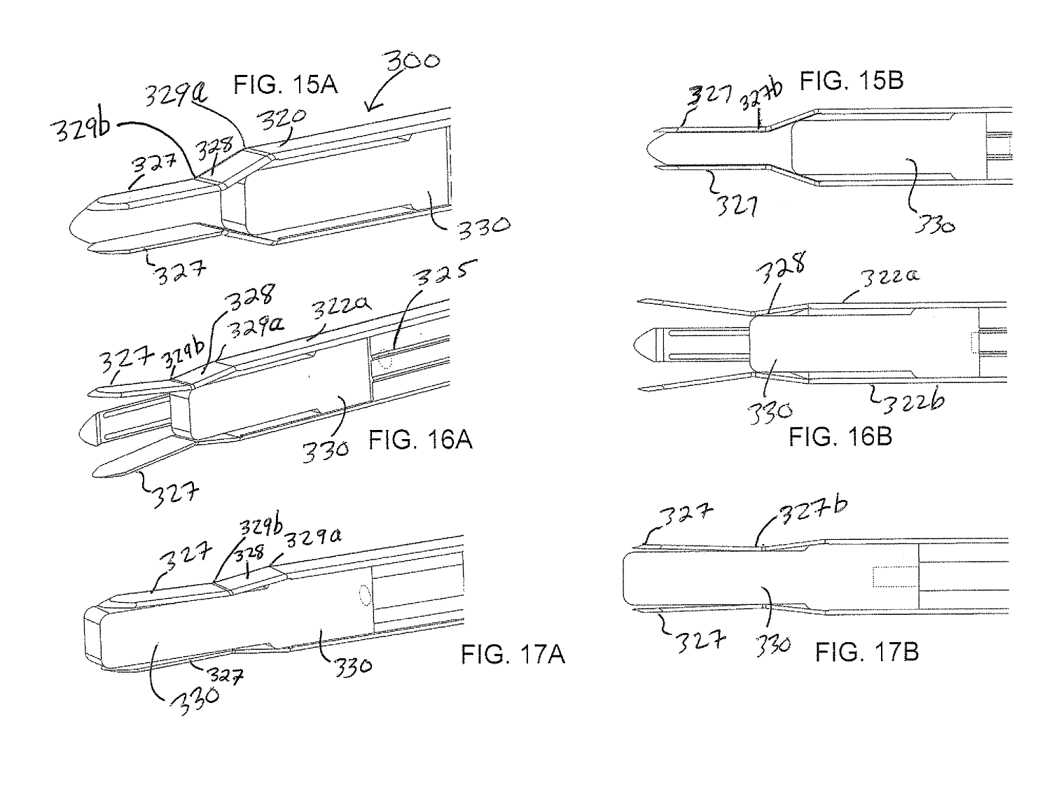

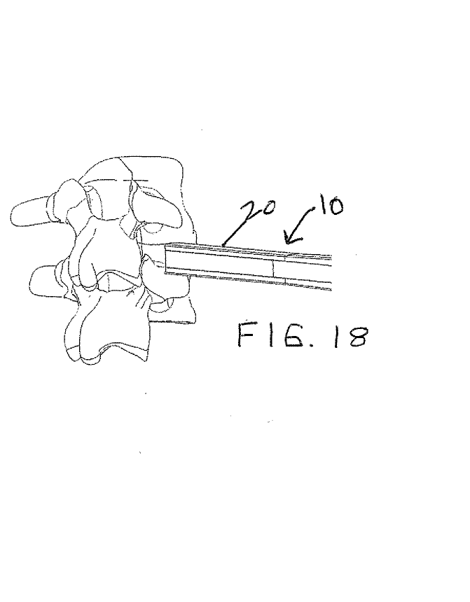

In a still further preferred embodiment, FIGS. 15A-B shows a tissue separator 300 comprising a retractor 320 and a dilator 330. The upper and lower guide surface portions 322, 324 of the retractor 320 may comprise flaps 327 that may be situated distal to a hinge 328 of the retractor 320. Hinge 328 may be integrally joined to the retractor arms 322a, 322b and may extend from an integral joint 329a on the retractor arms to a further joint 329b adjacent a base 327b (FIG. 17B) of flaps 327. In a further preferred embodiment (not shown) flaps 327 may extend directly from integral joint 329a, As shown in FIGS. 15A-B, flaps 327 are in a constricted position. As shown in FIG. 16A-B, when dilator 330 is inserted and in a partially forward position, dilator 330 bumps into hinge 328 which moves flaps 327 outward. FIGS. 17A-B depicts the position where the dilator 330 has been moved even further forward. In this case flaps 327 have reverted to an almost constricted position due to pressure from the tissue. As can be seen from FIGS. 15A-17B, hinge 328 on the surface of the retractor may be a double hinge comprising two or more joints (integral joints 329a, 329b) and the area between them.

As shown in FIG. 16A, the medial wall 325 of the retractor 320, which is the rear wall of the tissue separator 300 spanning the top and bottom surfaces of the retractor 320, may preferably be reinforced all along its length.

The flaps 327 may be made of any suitable material including, but not limited to, metal or metal alloys (collectively metallic material) and biocompatible plastics or other polymers.

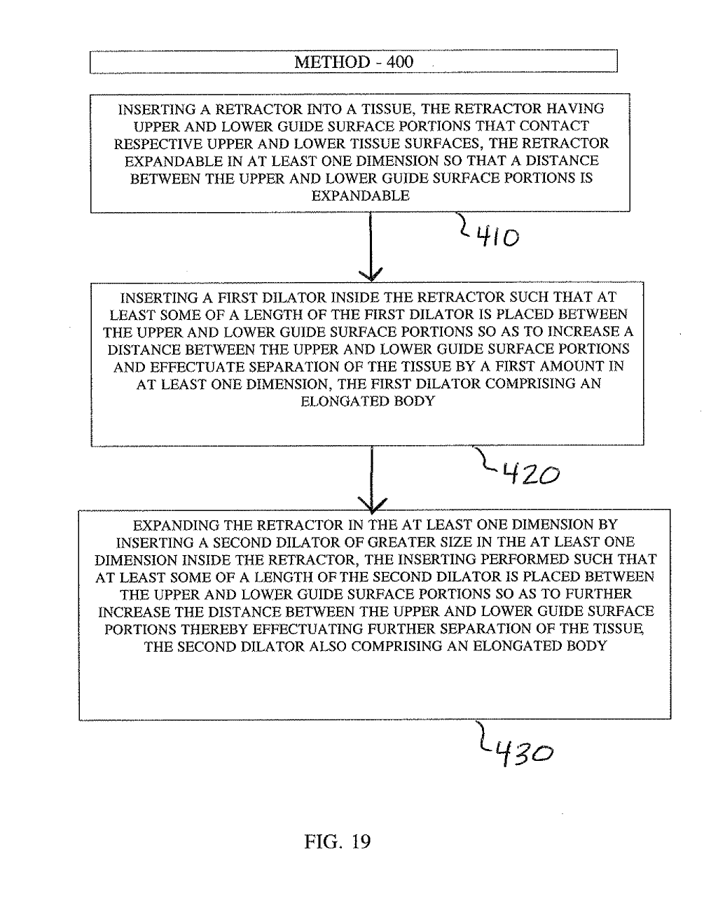

As shown in FIG. 19, the present invention may also be described as a method 400 of tissue separation. Method 400 may comprise a step 410 of inserting a retractor into a tissue, the retractor having upper and lower guide surface portions that contact respective upper and lower tissue surfaces, the retractor expandable in at least one dimension so that a distance between the upper and lower guide surface portions is expandable.

Method 400 may also include a step 420 of inserting a first dilator inside the retractor such that at least some of a length of the first dilator is placed between the upper and lower guide surface portions so as to increase a distance between the upper and lower guide surface portions and effectuate separation of the tissue by a first amount in at least one dimension. The first dilator may utilize any of the embodiments described for the apparatus of the present invention and may comprise an elongated body.

A further step 430 of method 400 may be expanding the retractor in the at least one dimension by inserting a second dilator of greater size in the at least one dimension inside the retractor, the inserting performed such that at least some of a length of the second dilator is placed between the upper and lower guide surface portions so as to further increase the distance between the upper and lower guide surface portions thereby effectuating further separation of the tissue by a greater distance. The second dilator may utilize any of the embodiments described for the apparatus of the present invention and may comprise an elongated body. In a preferred embodiment of method 400, the first and further dilators may be inserted into the retractor without the first and further dilators contacting the upper and lower tissue surfaces. The method 400 also may involve effectuating still further dilation by successively inserting one or more further dilators (beyond the second dilator) between the upper and lower guide surface portions so as to further increase the distance between the upper and lower guide surface portions by successively greater amounts. The number of such further dilators may be one, at least one, two, at least two, three, at least three, four, at least four, five, at least five, or a higher number, as needed. In some preferred embodiments of method 400 the expanding the retractor further includes successively removing a dilator before successively inserting the second dilator (or the further dilator).

Method 400 may utilize any of the embodiments of tissue separator 10, 100, 200, 300. For example, in one preferred embodiment, inserting the retractor into a tissue involves inserting opposing upper and lower U-shaped walls into the tissue. In another example, the second dilator (and any further dilator) may comprise a core element and a support element, and the expanding the retractor may comprise first replacing the core element of a first size of the first dilator with a core element of a second size of the second dilator, the second size core element larger than the first size core element, and then afterwards replacing the support element of a first size of the first dilator with a support element of a second size of the second dilator, the second size support element larger than the first size support element. In a further example, each dilator (and any further dilator) may comprise a core element and a support clement, and wherein the expanding the retractor comprises removing the core element of a particular dilator (for example the first dilator) before removing the support element of that particular dilator (for example the first dilator). In a still further example, the first and second dilators each comprise a core element and a support element, and wherein the expanding the retractor comprises inserting a core element of the second dilator before removing the support element of the first dilator.

In another example, inserting the core element of the second dilator may be preceded by removing the core element of the first dilator. In a further example, each of the first dilator and second dilator may comprise a core element and a support element, and a multiple expansion of the retractor may comprise first expanding the retractor by removing the core element of the first dilator, inserting the core element of the second dilator of a second size before removing the support element of the first dilator, removing the support element of the first dilator, inserting the support element of the second dilator, and then further expanding the retractor including by removing the core element of the second dilator and inserting a core element of a third dilator before removing the support element of the second dilator. The process between the second and third dilators may be continued similar to the process between the first and second dilators. The core element is replaced before the support element is replaced.

In still another example, the method 400 may involve using a hinge to move the upper and lower guide surface portions to an initial distance prior to inserting the first dilator, the hinge also setting a maximum retraction of the upper and lower guide surface portions for the first dilator. The method 400 may also involve using the hinge to move the upper and lower guide surface portions to an initial distance prior to inserting the second dilator, the hinge also setting a maximum retraction of the upper and lower guide surface portions for the second dilator.

In a further example, method 400 may have a further step of configuring the upper and lower guide surface portions to converge at distal tips at an initial position and to be expanded by insertion of the first and second dilators.

In some preferred embodiments, method 400 may also comprise a step wherein the first and second dilators each move flaps at the upper and lower guide surface portions of the retractor.

In other preferred embodiments of the method of the present invention other than method 400, the insertion of the second. dilator (or further dilators) may occur without removal of the previously inserted first dilator (or second or further dilators). For example, in such a method of tissue separation, a first step may involve inserting a retractor into a tissue, the retractor having upper and lower guide surface portions that contact respective upper and lower tissue surfaces, the retractor expandable in at least one dimension so that a distance between the upper and lower guide surface portions is expandable.

A second step may involve inserting a first dilator inside the retractor such that at least some of a length of the first dilator is placed between the upper and lower guide surface portions so as to increase a distance between the upper and lower guide surface portions and effectuate separation of the tissue by a first distance. The first dilator may comprise an elongated body.

A third step may be expanding the retractor in at least one dimension by inserting a second dilator of greater size in the at least one dimension inside the retractor without removing previously inserted first dilator. This may be accomplished in a variety of ways, for example by using dilators whose outer walls are flexible and hollow. The inserting may be performed such that at least some of a length of the second dilator is placed between the upper and lower guide surface portions so as to further increase the distance between the upper and lower guide surface portions thereby effectuating further separation of the tissue by a greater distance. The second dilator may also comprise an elongated body.

In a still another preferred embodiment of a method of the present invention, the method of sizing a limit of tissue separation may comprise a first step of (a) assessing a quality of tissue being separated by

(i) inserting a retractor into a tissue, the retractor having upper and lower guide surface portions that contact respective upper and lower tissue surfaces, the retractor expandable in at least one dimension so that a distance between the upper and lower guide surface portions is expandable, and by

(ii) inserting a first dilator inside the retractor such that at least some of a length of the first dilator is placed between the upper and lower guide surface portions so as to increase a distance between the upper and lower guide surface portions and effectuate separation of the tissue by a first distance.

A second step may involve (b) sizing/assessing a limit to which the tissue is separable by expanding the retractor in at least one dimension by successively inserting a further dilator of successively greater size in the at least one dimension inside the retractor, the inserting performed such that at least some of a length of each of the further dilators is placed between the upper and lower guide surface portions so as to further increase the distance between the upper and lower guide surface portions thereby effectuating further separation of the tissue by successively greater distances. In some preferred versions, the second step includes removing a dilator before a further dilator is inserted.

The present invention may also be characterized as a method of using a retractor for an insertion of an element into a human body. This method may comprise a step of inserting a retractor into a tissue, the retractor having upper and lower guide surface portions that contact respective upper and lower tissue surfaces, the retractor expandable in at least one dimension so that a distance between the upper and lower guide surface portions is expandable. This method may also include a further step of inserting a dilator inside the retractor such that at least some of a length of the first dilator is placed between the upper and lower guide surface portions so as to increase a distance between the upper and lower guide surface portions and effectuate separation of the tissue by a first amount in at least one dimension. A further step of the method may be forming a passageway inside the retractor by removing the dilator. The method may also involve a step of inserting (i) an implant, (ii) a surgical tool or (iii) an implant and a surgical tool, through the passageway and into the human body.

In any embodiment of the present invention, the surface guide portions, for example the upper and lower surface guide portions, may have teeth/ridges/protrusions externally on the ends that may be embedded into the tissue (endplate or other) to provide at least some fixation of the retractor to the tissue. The upper and lower surface guide portions may have internally facing protrusions (stoppers) to prevent dilators or other instruments from advancing beyond the tip of the retractor.

The upper and lower guide surface portions typically conform to the surface of the tissue against which they are pressed as the dilator advances, remaining substantially parallel to each other. Optionally, the thickness of the guide portions may be variable so as to define a single or more preferably double hinge arrangement, with the distal part of the guide portion being relatively more rigid, and being forced outwards as a unit, thereby spreading the applied pressure more evenly across a relatively large area of tissue.

Where dilation is required in two perpendicular dimensions, two pairs of guide surface portions may be used, and successive dilators may increase, either simultaneously or intermittently, in both dimensions. Optionally, a single sequence of dilators may be used with each being inserted in two orientations to separately increase both height and width dimensions.

The apparatus of the present invention can be a universal tool that incorporates other inventive concepts. For example, if the apparatus is used according to the “NonLinear” concept (i.e., as a part of a system and method as described in various prior co-assigned patent applications) in which the tools and implants are deflected into circuitous trajectories, the medial wall of the retractor may be reinforced to enable curling of the multi-link structures around the distal end of the wall.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. An apparatus for tissue separation, comprising:
a hollow retractor expandable in at least one dimension, the retractor having upper and lower guide surface portions for contacting respective upper and lower tissue surfaces;
a first dilator having a distal portion configured to fit inside the upper and lower guide surface portions of the retractor so as to expand a distance between the upper and lower guide surface portions of the retractor and thereby separate the upper and lower tissue surfaces by a first distance; and
a second dilator having a distal portion configured to fit inside the upper and lower guide surface portions of the retractor so as to expand a distance between the upper and lower guide surface portions of the retractor and thereby render the upper and lower tissue surfaces being separated by a second distance, the second distance greater than the first distance.

2. The apparatus of claim 1, further comprising the retractor deformable in at least the upper and lower guide surface portions of the retractor to accommodate dilators that are successively larger in at least one dimension.

3. The apparatus of claim 1, further comprising the at least a distal portion of the length of the retractor made from superelastic material so as to be smoothly deformable.

4. The apparatus of claim 1, wherein the retractor comprises upper and lower U-shaped walls.

5. The apparatus of claim 4, wherein each of the first and second dilators comprise a core element and a support element, the support element located on a side of the core element.

6. The apparatus of claim 1, wherein each of the first and second dilators comprise a core element having a top surface and a bottom surface, and a support element having at least one side wall, the core element removable from the retractor without the support element having to be removed from the retractor.

7. The apparatus of claim 1, further comprising a core element of a second dilator, the core element having a top surface and a bottom surface, and a support element of a first dilator, the first dilator smaller in size than the second dilator, the support element having at least one side wall.

8. The apparatus of claim 1, further comprising the first dilator having a first core element having a top and bottom surface and a first support element having at least one side wall and having a first size for the first core element and first support element and comprising the second dilator having a second core element having a top and bottom surface and a second support element having at least one side wall and having a second size for the second core element and second support element, the second size larger than the first size in at least one dimension.

9. The apparatus of claim 1, further comprising a cross-sectional shape of the upper and lower guide surface portions extending across a majority of a length of the retractor.

10. The apparatus of claim 1, further comprising the retractor has a generally rectangular cross-section at a distal end.

11. The apparatus of claim 1, wherein the retractor is expandable in a height dimension.

12. The apparatus of claim 1, wherein the retractor is expandable in a height dimension and in a width dimension.

13. The apparatus of claim 1, wherein the guide surface portions are comprised of opposing guide surfaces.

14. The apparatus of claim 1, further comprising tips of the upper and lower guide surface portions of the retractor are urged to contact one another before insertion of the first or second dilator.

15. The apparatus of claim 1, further comprising the retractor including upper and lower spring arms that converge at a tip before insertion of the first or second dilator, the tip comprising part of the upper and lower guide surface portions.

16. The apparatus of claim 1, further comprising a hinge mechanism at a proximal end of the retractor for initial retraction of the upper and lower guide surface portions.

17. The apparatus of claim 16, further comprising the hinge mechanism controlled by a screw mechanism, the screw mechanism also setting a maximum retraction of the upper and lower guide surface portions that is possible for a particular first or further dilator.

18. The apparatus of claim 1, further comprising the retractor having a hinge, wherein the upper and lower guide surface portions comprise flaps, the flaps distal to the hinge.

19. The apparatus of claim 18, further comprising the flaps extend from an integral joint of a double hinge on the retractor.

20. The apparatus of claim 1, wherein the upper and lower guide surface portions are parallel to one another.

21. A method of tissue separation, comprising:
inserting a retractor into a tissue, the retractor having upper and lower guide surface portions that contact respective upper and lower tissue surfaces, the retractor expandable in at least one dimension so that a distance between the upper and lower guide surface portions is expandable;
inserting a first dilator inside the retractor such that at least some of a length of the first dilator is placed between the upper and lower guide surface portions so as to increase a distance between the upper and lower guide surface portions and effectuate separation of the tissue by a first amount in at least one dimension, the first dilator comprising an elongated body;
expanding the retractor in the at least one dimension by successively inserting a second dilator of greater size in the at least one dimension inside the retractor, the inserting performed such that at least some of a length of the second dilator is placed between the upper and lower guide surface portions so as to further increase the distance between the upper and lower guide surface portions thereby effectuating further separation of the tissue, the second dilator also comprising an elongated body.

22. The method of claim 21, further comprising the expanding the retractor includes removing a dilator before inserting the second dilator.

23. The method of claim 21, further comprising the second dilator expanding the retractor in a cephalad-caudad dimension.

24. The method of claim 21, further comprising the first and second dilators inserted into the retractor without the first and second dilators contacting the upper and lower tissue surfaces.

25. The method of claim 21, further comprising inserting the retractor into a tissue involves inserting opposing upper and lower U-shaped walls into the tissue.

26. The method of claim 21, further comprising each of the first and second dilators comprise a core element and a support element, and the expanding the retractor comprises first replacing the core element of a first size of the first dilator with a core element of a second size of the second dilator, the second size core element larger than the first size core element, and then afterwards replacing the support element of a first size of the first dilator with a support element of a second size of the second dilator, the second size support element larger than the first size support element.

27. The method of claim 21, further comprising the first dilator and the second dilator each comprise a core element and a support element, and wherein the expanding the retractor comprises removing the core element of the first dilator before removing the support element of the first dilator.

28. The method of claim 21, further comprising the first dilator and the second dilator each comprise a core element and a support element, and wherein the expanding the retractor comprises inserting a core element of the second dilator before removing a support element of the first dilator.

29. The method of claim 28, further comprising inserting the core element of the second dilator is preceded by removing the core element of the first dilator.

30. The method of claim 21, further comprising the first dilator and the second dilator each comprise a core element and a support element, and wherein a multiple expansion of the retractor comprises first expanding the retractor by removing the core element of the first dilator, inserting a core element of the second dilator of a second size before removing a support element of the first dilator, removing the support element of the first dilator, inserting a support element of the second dilator, and then further expanding the retractor including by removing the core element of the second dilator and inserting a core element of a third dilator before removing the support element of the second dilator.

31. The method of claim 21, further comprising using a hinge to move the upper and lower guide surface portions to an initial distance prior to inserting the first dilator, the hinge also setting a maximum retraction of the upper and lower guide surface portions for the first dilator.

32. The method of claim 21, further comprising configuring the upper and lower guide surface portions to converge at distal tips at an initial position and to be expanded by insertion of the first and second dilators.

33. The method of claim 21, further comprising the first and second dilators each moving flaps at the upper and lower guide surface portions of the retractor.

34. A method of tissue separation, comprising:
inserting a retractor into a tissue, the retractor having upper and lower guide surface portions that contact respective upper and lower tissue surfaces, the retractor expandable in at least one dimension so that a distance between the upper and lower guide surface portions is expandable;
inserting a first dilator inside the retractor such that at least some of a length of the first dilator is placed between the upper and lower guide surface portions so as to increase a distance between the upper and lower guide surface portions and effectuate separation of the tissue by a first distance, the first dilator comprising an elongated body;
expanding the retractor in at least one dimension by inserting a second dilator of greater size in the at least one dimension inside the retractor without removing the first dilator, the inserting performed such that at least some of a length of the second dilator is placed between the upper and lower guide surface portions so as to further increase the distance between the upper and lower guide surface portions thereby effectuating further separation of the tissue by a greater distance.

35. A method of sizing a limit of tissue separation, comprising:
(a) assessing a quality of tissue being separated by
(i) inserting a retractor into a tissue, the retractor having upper and lower guide surface portions that contact respective upper and lower tissue surfaces, the retractor expandable in at least one dimension so that a distance between the upper and lower guide surface portions is expandable, and by
(ii) inserting a first dilator inside the retractor such that at least some of a length of the first dilator is placed between the upper and lower guide surface portions so as to increase a distance between the upper and lower guide surface portions and effectuate separation of the tissue by a first distance; and
(b) sizing/assessing a limit to which the tissue is separable by
expanding the retractor in at least one dimension by successively inserting a further dilator of successively greater size in the at least one dimension inside the retractor, the inserting performed such that at least some of a length of each of the further dilators is placed between the upper and lower guide surface portions so as to further increase the distance between the upper and lower guide surface portions thereby effectuating further separation of the tissue in successively greater amounts.

36. An apparatus for tissue separation, comprising:
a hollow retractor expandable in at least one dimension, the retractor having upper and lower guide surface portions for contacting respective upper and lower tissue surfaces;
a dilator having a distal portion configured to fit inside the upper and lower guide surface portions of the retractor so as to expand a distance between the upper and lower guide surface portions of the retractor and thereby separate the upper and lower tissue surfaces by a first distance.

37. The apparatus of claim 36, further comprising a mechanism for maintaining the hollow retractor in a retracted position with a set distance between the upper and lower guide surface portions when the dilator is removed before being replaced with a dilator of a different size.

38. The apparatus of claim 37, wherein the mechanism is a hinge mechanism at a proximal end of the retractor for fixing a position of one or more retractor arms.

39. The apparatus of claim 37, wherein the mechanism is comprised of the retractor arms having upper and lower U-shaped walls and the dilator having a core element that has a top and bottom surface and a support element that has at least one side wall, the support element located on a side of the core element.

40. The apparatus of claim 39, further comprising the core element removable from the retractor without the support element having to be removed from the retractor.

41. A method of using a retractor for an insertion of an element into a human body, comprising:
inserting a retractor into a tissue, the retractor having upper and lower guide surface portions that contact respective upper and lower tissue surfaces, the retractor expandable in at least one dimension so that a distance between the upper and lower guide surface portions is expandable;
inserting a dilator inside the retractor such that at least some of a length of the first dilator is placed between the upper and lower guide surface portions so as to increase a distance between the upper and lower guide surface portions and effectuate separation of the tissue by a first amount in at least one dimension;
forming a passageway inside the retractor by removing the dilator; and
inserting an implant through the passageway and into the human body.

42. A method of using a retractor for an insertion of an element into a human body, comprising:
inserting a retractor into a tissue, the retractor having upper and lower guide surface portions that contact respective upper and lower tissue surfaces, the retractor expandable in at least one dimension so that a distance between the upper and lower guide surface portions is expandable;
inserting a dilator inside the retractor such that at least some of a length of the first dilator is placed between the upper and lower guide surface portions so as to increase a distance between the upper and lower guide surface portions and effectuate separation of the tissue by a first amount in at least one dimension;
forming a passageway inside the retractor by removing the dilator; and
inserting a surgical tool through the passageway and into the human body.
